(19) United States
(12) Patent Application Publication
(10) Pub. No.: US 2002/0062529 A1
(43) Pub. Date: May 30, 2002

(54) Dye composition containing 1,8-bis(2,5-diaminophenoxy)-3,6-dioxaoctane, an additional oxidation base and a coupler, and dyeing processes

(75) Inventor: Marie-Pascale Audousset, Asnieres (FR)

Correspondence Address: FINNEGAN, HENDERSON, FARABOW, GARRETT &, DUNNER LLP, 1300 I STREET, NW, WASHINGTON, DC, 20005, US

(73) Assignee: L'Oreal S.A.

(21) Appl. No.: 09/829,433

(22) Filed: Apr. 10, 2001

Related U.S. Application Data
(63) Continuation-in-part of application No. 09/335,742, filed on Jun. 18, 1999.

(30) Foreign Application Priority Data
Jun. 19, 1998 (FR) 98 07793

(51) Int. Cl. A61K 7/13

(52) U.S. Cl. 8/405; 8/406; 8/410; 8/415; 8/421

(57) ABSTRACT

The invention relates to a composition for the oxidation dyeing of keratin fibers, containing a first oxidation base chosen from 1,8-bis(2,5-diaminophenoxy)-3,6-dioxaoctane and acid-addition salts thereof, at least one second selected oxidation base and at least one coupler; as well as to the oxidation dyeing process using this composition and multicompartment devices/kits.

The invention relates to a composition for the oxidation dyeing of keratin fibers, containing a first oxidation base chosen from 1,8-bis(2,5-diaminophenoxy)-3,6-dioxaoctane, and the acid-addition salts thereof, at least one second selected oxidation base and at least one coupler; as well as to the oxidation dyeing process using this composition.

It is known practice to dye keratin fibers, and in particular human hair, with dye compositions containing oxidation dye precursors, in particular ortho- or para-phenylenediamines, ortho- or para-aminophenols, bis(phenyl)alkylenediamines or heterocyclic compounds, which are generally referred to as oxidation bases. Oxidation dye precursors, or oxidation bases, are colorless or weakly colored compounds which, when combined with oxidizing products, can give rise to colored and coloring compounds by a process of oxidative condensation.

It is also known that the shades obtained with these oxidation bases can be varied by combining them with couplers, Le., coloration modifiers. These couplers or coloration modifiers are chosen in particular from aromatic meta-diamines, meta-aminophenols, meta-diphenols and certain heterocyclic compounds.

The variety of molecules used as oxidation bases and couplers allows a wide variety of colors to be obtained.

The so-called “permanent” coloration obtained by means of these oxidation dyes should moreover satisfy a certain number of desired objectives. Thus, it is desired to have no toxicological drawbacks, to obtain shades of the desired intensity and to show good resistance to external agents (light, bad weather, washing, permanent-waving, perspiration and friction).

The dyes should also allow grey hairs to be covered, and, lastly, they should be as unselective as possible, i.e. they should give the smallest possible differences in coloration along the same keratin fiber, which may in fact be differently sensitized (i.e. damaged) between its tip and its root.

It has already been proposed, in particular in patent application WO 92/13824, to use 2,5-diaminophenoxyoxaalkanes as oxidation bases and optionally in the presence of a coupler. However, the colorations obtained using these oxidation bases are not entirely satisfactory, in particular as regards the intensity of the colorations obtained and their resistance to the various treatments to which the hair may be subjected. The colorations obtained in particular show insufficient resistance to the action of light.

The inventor has now discovered, entirely surprisingly and unexpectedly, that the combination of 1,8-bis(2,5-diaminophenoxy)-3,6-dioxaoctane, and/or of at least one of the acid-addition salts thereof, with at least one second suitably selected oxidation base and at least one coupler, can give intense colorations which moreover can have improved properties of resistance with respect to the various attacking factors to which the hair may be subjected (shampooing, light, bad weather, permanent-waving, perspiration, friction, etc.).

These discoveries form the basis of the present invention.

A first subject of the invention is thus a composition for the oxidation dyeing of keratin fibers, and in particular human keratin fibers such as the hair, comprising:

at least one first oxidation base chosen from 1,8-bis(2,5-diaminophenoxy)-3,6-dioxaoctane and acid-addition salts thereof,

at least one second oxidation base chosen from para-phenylenediamine, para-toluenediamine, N,N-bis-(β-hydroxyethyl)-para-phenylenediamine, 2-(β-hydroxyethyl)-para-phenylenediamine, 2,6-dimethyl-para-phenylenediamine, 2-isopropyl-para-phenylenediamine, 2-chloro-para-phenylenediamine, N-phenyl-para-phenylenediamine, 4,4′-diaminodiphenylamine, N-methoxyethyl-para-phenylenediamine, 2-n-propyl-para-phenylenediamine, 4-aminophenol, N-methyl-4-aminophenol, 2-hydroxymethyl-4-aminophenol, 3-methyl-4-aminophenol, 2-aminomethyl-4-aminophenol, 2-(β-hydroxyethylaminomethyl)-4-aminophenol, 2-methoxy-4-aminophenol, 2-methoxymethyl-4-aminophenol, tetraaminopyrimidine, 4-hydroxy-2,5,6-triaminopyrimidine, 4,5-diamino-1-ethyl-3-methylpyrazole, 4,5-diamino-N-methylpyrazole, 4,5-diamino-1-(4′-chlorobenzyl)pyrazole, N,N′-bis(β-hydroxyethyl)-N,N′-bis(4′-aminophenyl)-1,3-diaminopropanol, 3-amino-6-dimethylaminopyridine and pyrazolo[1,5-a]pyrimidines of formula (I):

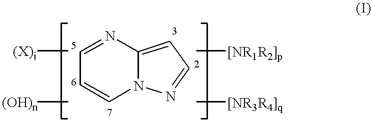

in which:

R₁, R₂, R₃ and R₄, which are identical or different, are chosen from a hydrogen atom, C₁-C₄ alkyl radicals, aryl radicals, C₁-C₄ hydroxyalkyl radicals, C₂-C₄ polyhydroxyalkyl radicals, (C₁-C₄)alkoxy(C₁-C₄)alkyl radicals, C₁-C₄ aminoalkyl radicals wherein said amino can be protected with a protective group chosen from acetyl, ureido and sulphonyl groups, (C₁-C₄)alkylamino(C₁-C₄)alkyl radicals, di((C₁-C₄)alkyl)amino(C₁-C₄)alkyl radicals, wherein said dialkyls can form a ring chosen from 5- and 6-membered aliphatic and heterocyclic rings, hydroxy(C₁-C₄)alkylamino(C₁-C₄)alkyl radicals, and di(hydroxy(C₁-C₄)alkyl)amino-(C₁-C₄)alkyl radicals;

radicals X, which are identical or different, and are chosen from a hydrogen atom, C₁-C₄ alkyl radicals, aryl radicals, C₁-C₄ hydroxyalkyl radicals, C₂-C₄ polyhydroxyalkyl radicals, C₁-C₄ aminoalkyl radicals, (C₁-C₄)alkylamino(C₁-C₄)alkyl radicals, di((C₁-C₄)alkyl)amino(C₁-C₄)alkyl radicals, wherein said dialkyls can form a ring chosen from 5- and 6-membered aliphatic and heterocyclic rings), hydroxy(C₁-C₄)alkyl- and di(hydroxy(C₁-C₄)alkyl)amino(C₁-C₄)alkyl radicals, amino radicals, (C₁-C₄)alkyl- and di((C₁-C₄)alkyl)amino radicals, halogen atoms, carboxylic acid groups and sulphonic acid groups;

i is chosen from 0, 1, 2 or 3;

p is chosen from 0 or 1;

q is chosen from 0 or 1;

n is chosen from 0 or 1;

with the proviso that:

(i) the sum p+q is other than 0;

(ii) when p+q is equal to 2, then n is 0 and the groups NR₁R₂ and NR₃R₄ occupy positions (2,3); (5,6); (6,7); (3,5) or (3,7);

(iii) when p is equal to 1 and q is equal to 0, then n is 1 and the group NR₁R₂ and the OH group occupy positions (2,3); (5,6); (6,7); (3,5) or (3,7);

(iv) when p is equal to 0 and q is equal to 1, then n is 1 and the group NR₃R₄ and the OH group occupy positions (2,3); (5,6); (6,7); (3,5) or (3,7);

and acid-addition salts thereof;

and at least one coupler.

As mentioned above, the dye composition in accordance with the invention can give intense colorations which moreover can have excellent properties of resistance with respect to the action of various external agents (light, bad weather, washing, permanent-waving, perspiration, friction). These properties can be particularly noteworthy as regards the light-fastness of the colorations obtained.

Representative pyrazolo[1,5-a]pyrimidines of formula (I), which can be used as the at least one second oxidation base in the dye compositions in accordance with the invention, include:

pyrazolo[1 ,5-a]pyrimidine-3,7-diamine;

2-methylpyrazolo[1,5-a]pyrimidine-3,7-diamine;

2,5-dimethylpyrazolo[1,5-a]pyrmidine-3,7-diamine;

pyrazolo[1,5-a]pyrimidine-3,5-diamine;

2,7-dimethylpyrazolo[1,5-a]pyrimidine-3,5-diamine;

3-aminopyrazolo[1,5-a]pyrimidin-7-ol;

3-amino-5-methylpyrazolo[1,5-a]pyrimidin-7-ol;

3-aminopyrazolo[1,5-a]pyrimidin-5-ol;

2-(3-aminopyrazolo[1,5-a]pyrimidin-7-ylamino)ethanol;

3-amino-7-β-hydroxyethylamino-5-methylpyrazolo[1,5-a]pyrimidine;

2-(7-aminopyrazolo[1,5-a]pyrimidin-3-ylamino)ethanol;

2-((3-aminopyrazolo[1,5-a]pyrimidin-7-yl)-(2-hydroxyethyl)amino)ethanol;

2-((7-aminopyrazolo[1,5-a]pyrimidin-3-yl)-(2-hydroxyethyl)amino)ethanol;

5,6-dimethylpyrazolo[1,5-a]pyrimidine-3,7-diamine;

2,6-dimethylpyrazolo[1,5-a]pyrimidine-3,7-diamine;

2,5, N-7, N-7-tetramethylpyrazolo[1,5-a]pyrimidine-3,7-diamine;

and acid-addition salts thereof.

The nature of the at least one coupler which can be used in the dye composition in accordance with the invention is not critical. They can be chosen from the couplers conventionally used for dyeing keratin fibers, such as meta-phenylenediamines, meta-aminophenols, meta-diphenols and heterocyclic couplers including, for example, indoles, indolines, pyridines, and pyrazolones, and acid-addition salts thereof.

Representative couplers include 5-amino-2-methylphenol, 5-N-(β-hydroxyethyl)amino-2-methylphenol, 3-aminophenol, 1,3-dihydroxybenzene, 1,3-dihydroxy-2-methylbenzene, 4-chloro-1,3-dihydroxybenzene, 2,4-diamino-1-(β-hydroxyethyloxy)benzene, 2-amino-4-(β-hydroxyethylamino)-1 -methoxybenzene, 1,3-diaminobenzene, 1,3-bis(2,4-diaminophenoxy)propane, sesamol, α-naphthol, 6-hydroxyindole, 4-hydroxyindole, 4-hydroxy-N-methylindole, 6-hydroxyindoline, 6-hydroxybenzomorpholine, 2,6-dihydroxy-4-methylpyridine, 1 -H-3-methylpyrazol-5-one and 1-phenyl-3-methylpyrazol-5-one, and acid-addition salts thereof.

In general, representative acid addition salts used in the invention (oxidation bases and couplers) include hydrochlorides, hydrobromides, sulphates, citrates, succinates, tartrates, lactates and acetates.

The at least one first oxidation base chosen from 1,8-bis(2,5-diaminophenoxy)-3,5-dioxaoctane and acid-addition salts thereof preferably represent from 0.0005 to 12% by weight approximately relative to the total weight of the dye composition, and even more preferably from 0.005 to 6% by weight approximately relative to this weight.

The at least one second oxidation base in accordance with the invention and the acid-addition salts thereof preferably represent from 0.0005 to 12% by weight approximately relative to the total weight of the dye composition, and even more preferably from 0.005 to 6% by weight approximately relative to this weight.

The at least one coupler preferably represents from 0.0001 to 10% by weight approximately relative to the total weight of the dye composition, and even more preferably from 0.005 to 5% by weight approximately relative to this weight.

The medium which is suitable for dyeing (or support) generally comprises water or a mixture of water and at least one organic solvent to dissolve the compounds which would not be sufficiently water-soluble. As organic solvents, mention may be made, for example, of C₁-C₄ lower alkanols, such as ethanol and isopropanol; glycerol; glycols and glycol ethers such as 2-butoxyethanol, propylene glycol, propylene glycol monomethyl ether, diethylene glycol monoethyl ether and monomethyl ether, and aromatic alcohols such as benzyl alcohol or phenoxyethanol, similar products and mixtures thereof.

The solvents can be present in proportions preferably from 1 to 40% by weight approximately relative to the total weight of the dye composition, and even more preferably from 5 to 30% by weight approximately.

The pH of the dye composition in accordance with the invention is generally approximately from 3 to 12, and preferably approximately from 5 to 11. It can be adjusted to the desired value by means of acidifying or basifying agents usually used for dyeing keratin fibers.

Among the acidifying agents, mention may be made, for example, of inorganic or organic acids such as hydrochloric acid, orthophosphoric acid, sulphuric acid, carboxylic acids, such as acetic acid, tartaric acid, citric acid or lactic acid, and sulphonic acids.

Among the basifying agents, mention may be made, for example, of aqueous ammonia, alkaline carbonates, alkanolamines such as mono-, di- and triethanolamine and derivatives thereof, sodium hydroxide, potassium hydroxide and the compounds of formula (II) below:

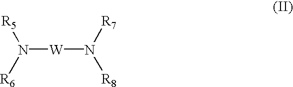

in which W is a propylene residue optionally having at least one substituent chosen from a hydroxyl group and C₁-C₆ alkyl radicals; R₅, R₆, R₇ and R₈, which may be identical or different, are chosen from a hydrogen atom and C₁-C₆ alkyl and C₁-C₆ hydroxyalkyl radicals.

The oxidation dye compositions in accordance with the invention can also contain at least one direct dye, in particular in order to modify the shades or to enrich them with glints.

The dye composition in accordance with the invention can also contain at least one of various adjuvants conventionally used in compositions for dyeing the hair, such as anionic, cationic, nonionic, amphoteric or zwitterionic surfactants or mixtures thereof, anionic, cationic, nonionic, amphoteric or zwitterionic polymers or mixtures thereof, inorganic or organic thickeners, antioxidants, penetrating agents, sequestering agents, fragrances, buffers, dispersing agents, conditioners such as, for example, volatile or non-volatile, modified or unmodified silicones, film-forming agents, ceramides, preserving agents, sunscreens, and opacifiers.

Needless to say, a person skilled in the art will take care to select optional complementary compounds such that the advantageous properties intrinsically associated with the oxidation dye composition in accordance with the invention are not, or are not substantially, adversely affected by the addition(s) envisaged.

The dye composition according to the invention can be in various forms, such as in the form of liquids, creams, gels or any other form which is suitable for dyeing keratin fibers, and in particular human hair.

A subject of the invention is also a process for the oxidation dyeing of keratin fibers, and in particular human keratin fibers such as the hair, using the dye composition as defined above.

According to an embodiment of this process, at least one dye composition as defined above is applied to the fibers, the color being developed at acidic, neutral or alkaline pH with the aid of an oxidizing agent which is added to the dye composition just at the time of use, or which is present in an oxidizing composition that is applied simultaneously or sequentially.

According to one preferred embodiment of the dyeing process of the invention, the dye composition described above is preferably mixed, at the time of use, with an oxidizing composition containing, in a medium which is suitable for dyeing, at least one oxidizing agent present in an amount which is sufficient to develop a coloration. The mixture obtained is then applied to the keratin fibers and is preferably left to stand on them for 3 to 50 minutes approximately, more preferably 5 to 30 minutes approximately, after which the fibers are rinsed, washed with shampoo, rinsed again and dried.

The oxidizing agent can be chosen from the oxidizing agents conventionally used for the oxidation dyeing of keratin fibers, such as hydrogen peroxide, urea peroxide, alkali metal bromates, persalts such as perborates and persulphates, and enzymes, among which mention may be made of peroxidases, 2-electron oxidoreductases such as uricases, and 4-electron oxygenases such as lactases. Hydrogen peroxide is particularly preferred.

The pH of the oxidizing composition containing the oxidizing agent as defined above is such that, after mixing with the dye composition, the pH of the resulting composition applied to the keratin fibers preferably ranges approximately from 3 to 12, and even more preferably from 5 to 11. It is adjusted to the desired value using acidifying or basifying agents usually used for dyeing keratin fibers, such as those defined above. The oxidizing composition as defined above can also contain at least one adjuvant conventionally used in compositions for dyeing the hair and as defined above.

The composition which is finally applied to the keratin fibers can be in various forms, such as in the form of liquids, creams, gels or any other form which is suitable for dyeing keratin fibers, and in particular human hair.

Another subject of the invention is a multi-compartment dyeing device, i.e., “kit” or any other multi-compartment packaging system, a first compartment of which contains the dye composition as defined above and a second compartment of which contains the oxidizing composition as defined above. These devices can be equipped with a means for dispensing the desired mixture onto the hair, such as the devices described in patent FR-2,586,913 in the name of L'Oreal, the disclosure of which is specifically incorporated by reference herein.

The examples which follow are intended to illustrate the invention without, however, being limiting in nature.

### EXAMPLES

### Examples 1 to 6 of Dyeing in Alkaline Medium

The dye compositions below, in accordance with the invention, were prepared (contents in grams):

| EXAMPLE | 1 | 2 | 3 | 4 | 5 | 6 |
|---|---|---|---|---|---|---|
| 1,8-Bis(2,5-diaminophenoxy)-3,6-dioxaoctane | 0.39 | 0.39 | 0.39 | 0.39 | 0.39 | 0.39 |
| tetrahydrochloride monohydrate | | | | | | |
| para-Phenylenediamine (second oxidation base) | 0.162 | — | 0.162 | — | — | — |
| para-Aminophenol (second oxidation base) | — | 0.163 | — | — | — | — |
| 3-Methyl-4-aminophenol (second oxidation base) | — | — | — | 0.184 | — | — |
| 2-(β-Hydroxyethyl)para-phenylenediamine | — | — | — | — | 0.337 | — |
| dihydrochloride (second oxidation base) | | | | | | |
| 2,6-Dimethyl-para-phenylenediamine dihydrochloride | — | — | — | — | — | 0.313 |
| (second oxidation base) | | | | | | |
| 5-N-(β-Hydroxyethyl)amino-2-methylphenol (coupler) | 0.498 | — | — | — | — | — |
| 2,4-Diaminophenoxyethanol dihydrochloride | — | 0.723 | — | — | — | — |
| (coupler) | | | | | | |
| 1,3-Dihydroxybenzene (coupler) | — | — | 0.33 | — | — | — |
| 5-Amino-2-methylphenol (coupler) | — | — | — | 0.369 | — | — |
| 3-Aminophenol | — | — | — | — | 0.327 | — |
| 6-Hydroxybenzomorpholine | — | — | — | — | — | 0.453 |
| Common dye support No. 1 | (*) | (*) | (*) | (*) | (*) | (*) |
| Demineralized water qs | 100 g | 100 g | 100 g | 100 g | 100 g | 100 g |

| (*) Common dye support No. 1: | | |
|---|---|---|
| 96° Ethyl alcohol | 18 | g |
| Sodium metabisulphite as an aqueous | 0.68 | g |
| solution at 35% | | |
| Pentasodium salt of diethylenetriamine- | 1.1 | g |
| pentaacetic acid | | |
| Aqueous ammonia containing 20% NH₃ | 10.0 | g |

Each of the above dye compositions was mixed weight for weight, at the time of use, with a 20-volumes hydrogen peroxide solution (6% by weight) of pH 3.

The mixture obtained was applied to locks of natural or permanent-waved grey hair containing 90% white hairs, for 30 minutes. The locks were then rinsed, washed with a standard shampoo, rinsed again and then dried.

The shades obtained are given in the table below:

| EXAM- | | Shade obtained on | Shade obtained on |
|---|---|---|---|
| PLE | Dyeing pH | natural hair | permanent-waved hair |
| 1 | 10 ± 0.2 | Violet iridescent light | Violet chestnut |
| | | chestnut | |
| 2 | 10 ± 0.2 | Violet-ash | Strong blue |
| 3 | 10 ± 0.2 | Iridescent golden dark | Iridescent golden light |
| | | blond | chestnut |
| 4 | 10 ± 0.2 | Violet iridescent blond | Ash-violet light chestnut |
| 5 | 10 ± 0.2 | Grey | Strong matt-grey |
| 6 | 10 ± 0.2 | Golden green | Light green |

### Examples 7 to 12 of Dyeing in Neutral Medium

The dye compositions below, in accordance with the invention, were prepared (contents in grams):

| EXAMPLE | 7 | 8 | 9 | 10 | 11 | 12 |
|---|---|---|---|---|---|---|
| 1,8-Bis(2,5-diaminophenoxy)-3,6-dioxaoctane | 0.39 | 0.39 | 0.39 | 0.39 | 0.39 | 0.39 |
| tetrahydrochloride monohydrate | | | | | | |
| para-Phenylenediamine (second oxidation base) | 0.162 | — | 0.162 | — | — | — |
| para-Aminophenol (second oxidation base) | — | 0.163 | — | — | — | — |
| 3-Methyl-4-aminophenol (second oxidation base) | — | — | — | 0.184 | — | — |
| 2-(β-Hydroxyethyl)para-phenylenediamine | — | — | — | — | 0.337 | — |
| dihydrochloride (second oxidation base) | | | | | | |
| 2,6-Dimethyl-para-phenylenediamine dihydrochloride | — | — | — | — | — | 0.313 |
| (second oxidation base) | | | | | | |
| 5-N-(β-Hydroxyethyl)amino-2-methylphenol (coupler) | 0.498 | — | — | — | — | — |
| 2,4-Diaminophenoxyethanol dihydrochloride | — | 0.723 | — | — | — | — |
| (coupler) | | | | | | |
| 1,3-Dihydroxybenzene (coupler) | — | — | 0.33 | — | — | — |
| 5-Amino-2-methylphenol (coupler) | — | — | — | 0.369 | — | — |
| 3-Aminophenol | — | — | — | — | 0.327 | — |
| 6-Hydroxybenzomorpholine | — | — | — | — | — | 0.453 |
| Common dye support No. 1 | (**) | (**) | (**) | (**) | (**) | (**) |
| Demineralized water qs | 100 g | 100 g | 100 g | 100 g | 100 g | 100 g |

| (**) Common dye support No. 2: | | |
|---|---|---|
| 96° Ethanol | 18 | g |
| K₂HPO₄/KH₂PO₄ (1.5 M/1 M) buffer | 10 | g |
| Sodium metabisulphite | 0.68 | g |
| Pentasodium salt of diethylenetriamine- | 1.1 | g |
| pentaacetic acid | | |

Each of the above dye compositions was mixed weight for weight, at the time of use, with a 20-volumes hydrogen peroxide solution (6% by weight) of pH 3.

The mixture obtained was applied to locks of natural or permanent-waved grey hair containing 90% white hairs, for 30 minutes. The locks were then rinsed, washed with a standard shampoo, rinsed again and then dried.

The shades obtained are given in the table below:

| | | | Shade |
|---|---|---|---|
| | | Shade | obtained on |
| | | obtained on | permanent- |
| EXAMPLE | Dyeing pH | natural hair | waved hair |
| 7 | 5.7 ± 0.2 | Violet light- | Strong violet- |
| | | chestnut | chestnut |
| 8 | 5.7 ± 0.2 | Ashen dark- | Strong ashen |
| | | bond | light chestnut |
| 9 | 5.7 ± 0.2 | Strong ash- | Strong grey |
| | | grey | |
| 10 | 5.7 ± 0.2 | Ash-grey | Violet ash- |
| | | | grey |
| 11 | 5.7 ± 0.2 | Blue-grey | Blue-grey |
| 12 | 5.7 ± 0.2 | Golden green | Green |

While the invention has been described in terms of various preferred embodiments and specific examples, those skilled in the art will recognize that various changes and modifications can be made without departing from the spirit and scope of the invention, as defined in the appended claims.

What is claimed is:

1. A composition for oxidation dyeing of keratin fibers, comprising:
at least one first oxidation base chosen from 1,8-bis(2,5-diaminophenoxy)-3,6-dioxaoctane and acid-addition salts thereof;
at least one second oxidation base chosen from para-phenylenediamine, para-toluenediamine, N,N-bis-(β-hydroxyethyl)-para-phenylenediamine, 2-(β-hydroxyethyl)-para-phenylenediamine, 2,6-dimethyl-para-phenylenediamine, 2-isopropyl-para-phenylenediamine, 2-chloro-para-phenylenediamine, N-phenyl-para-phenylenediamine, 4,4′-diaminodiphenylamine, N-methoxyethyl-para-phenylenediamine, 2-n-propyl-para-phenylenediamine, 4-aminophenol, N-methyl-4-aminophenol, 2-hydroxymethyl-4-aminophenol, 3-methyl-4-aminophenol, 2-aminomethyl-4-aminophenol, 2-(β-hydroxyethylaminomethyl)-4-aminophenol, 2-methoxy-4-aminophenol, 2-methoxymethyl-4-aminophenol, tetraaminopyrimidine, 4-hydroxy-2,5,6-triaminopyrimidine, 4,5-diamino-1-ethyl-3-methylpyrazole, 4,5-diamino-N-methylpyrazole, 4,5-diamino-1-(4′-chlorobenzyl)pyrazole, N,N′-bis(β-hydroxyethyl)-N,N′-bis(4′-aminophenyl)-1,3-diaminopropanol, 3-amino-6-dimethylaminopyridine and pyrazolo[1,5-a]pyrimidines of formula (I): 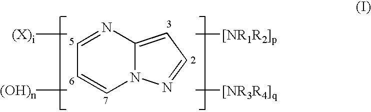
in which:
R₁, R₂, R₃ and R₄, which are identical or different, are chosen from a hydrogen atom, C₁-C₄ alkyl radicals, aryl radicals, C₁-C₄ hydroxyalkyl radicals, C₂-C₄ polyhydroxyalkyl radicals, (C₁-C₄)alkoxy(C₁-C₄)alkyl radicals, C₁-C₄ aminoalkyl radicals wherein said amino can be protected with a protective group chosen from acetyl, ureido and sulphonyl groups, (C₁-C₄)alkylamino(C₁-C₄)alkyl radicals, di((C₁-C₄)alkyl)amino(C₁-C₄)alkyl radicals, wherein said dialkyls can form a ring chosen from 5- and 6-membered aliphatic and heterocyclic rings, hydroxy(C₁-C₄)alkylamino(C₁-C₄)alkyl radicals, and di(hydroxy(C₁-C₄)alkyl)amino-(C₁-C₄)alkyl radicals;
radicals X are identical or different, and are chosen from a hydrogen atom, C₁-C₄ alkyl radicals, aryl radicals, C₁-C₄ hydroxyalkyl radicals, C₂-C₄ polyhydroxyalkyl radicals, C₁-C₄ aminoalkyl radicals, (C₁-C₄)alkylamino(C₁-C₄)alkyl radicals, di((C₁-C₄)alkyl)amino(C₁-C₄)alkyl radicals, wherein said dialkyls can form a ring chosen from 5- and 6-membered aliphatic and heterocyclic rings, hydroxy(C₁-C₄)alkylamino(C₁-C₄)alkyl radicals, di(hydroxy(C₁-C₄)alkyl)amino(C₁-C₄)alkyl radicals, amino radicals, (C₁-C₄)alkyl-amino radicals, di((C₁-C₄)alkyl)amino radicals, halogen atoms, carboxylic acid groups and sulphonic acid groups;
i is chosen from 0, 1, 2 and 3;
p is chosen from 0 and 1;
q is chosen from 0 and 1;
n is chosen from 0 and 1;
with the proviso that:
(i) the sum p+q is other than 0;
(ii) when p+q is equal to 2, then n is 0 and the groups NR₁R₂ and NR₃R₄ occupy positions (2,3); (5,6); (6,7); (3,5) and (3,7);
(iii) when p is equal to 1 and q is equal to 0, then n is 1 and the group NR₁R₂ and the OH group occupy positions (2,3); (5,6); (6,7); (3,5) and (3,7);
(iv) when p is equal to 0 and q is equal to 1, then n is 1 and the group NR₃R₄ and the OH group occupy positions (2,3); (5,6); (6,7); (3,5) and (3,7); and acid-addition salts thereof;
and at least one coupler.

2. The composition according to claim 1, wherein said keratin fibers are human keratin fibers.

3. The composition according to claim 2, wherein said human keratin fibers are hair.

4. The composition according to claim 1 wherein said
pyrazolo[1,5-a]pyrimidines of formula (I) are chosen from
pyrazolo[1,5-a]pyrimidine-3,7-diamine;
2-methylpyrazolo[1,5-a]pyrimidine-3,7-diamine;
2,5-dimethylpyrazolo[1,5-a]pyrimidine-3,7-diamine;
pyrazolo[1,5-a]pyrimidine-3,5-diamine;
2,7-dimethylpyrazolo[1,5-a]pyrimidine-3,5-diamine;
3-aminopyrazolo[1,5-a]pyrimidin-7-ol;
3-amino-5-methylpyrazolo[1,5-a]pyrimidin-7-ol;
3-aminopyrazolo[1,5-a]pyrimidin-5-ol;
2-(3-aminopyrazolo[1,5-a]pyrimidin-7-ylamino)ethanol;
3-amino-7-β-hydroxyethylamino-5-methylpyrazolo[1,5-a]pyrimidine;
2-(7-aminopyrazolo[1,5-a]pyrimidin-3-ylamino)ethanol;
2-((3-aminopyrazolo[1,5-a]pyrimidin-7-yl)-(2-hydroxyethyl)amino)ethanol;
2-((7-aminopyrazolo[1,5-a]pyrimidin-3-yl)-(2-hydroxyethyl)amino)ethanol;
5,6-dimethylpyrazolo[1,5-a]pyrimidine-3,7-diamine;
2,6-dimethylpyrazolo[1,5-a]pyrimidine-3,7-diamine;
2,5, N-7, N-7-tetramethylpyrazolo[1,5-a]pyrimidine-3,7-diamine;
and acid-addition salts thereof.

5. The composition according to claim 1, wherein said at least one coupler is chosen from meta-phenylenediamines, meta-aminophenols, meta-diphenols, heterocyclic couplers, and acid-addition salts thereof.

6. The composition according to claim 5, wherein said heterocyclic couplers are chosen from indoles, indolines, pyridines, pyrazolones, and acid-addition salts thereof.

7. The composition according to claim 5, wherein said at least one coupler is chosen from 5-amino-2-methylphenol, 5-N-(β-hydroxyethyl)amino-2-methylphenol, 3-aminophenol, 1,3-dihydroxybenzene, 1,3-dihydroxy-2-methylbenzene, 4-chloro-1,3-dihydroxybenzene, 2,4-diamino-1-(β-hydroxyethyloxy)benzene, 2-amino-4-(β-hydroxyethylamino)-1-methoxybenzene, 1,3-diaminobenzene, 1,3-bis(2,4-diaminophenoxy)propane, 6-hydroxyindole, 4-hydroxyindole, 4-hydroxy-N-methylindole, 6-hydroxyindoline, 6-hydroxybenzomorpholine, 2,6-dihydroxy-4-methylpyridine, 1-H-3-methylpyrazol-5-one, 1-phenyl-3-methylpyrazol-5-one, and acid-addition salts thereof.

8. The composition according to claim 1, wherein said at least one coupler is chosen from sesamol, α-naphthol, and acid-addition salts thereof.

9. The composition according to claim 1, wherein said acid-addition salts are chosen from hydrochlorides, hydrobromides, sulphates, citrates, succinates, tartrates, lactates and acetates.

10. The composition according to claim 4, wherein said acid-addition salts are chosen from hydrochlorides, hydrobromides, sulphates, citrates, succinates, tartrates, lactates and acetates.

11. The composition according to claim 5, wherein said acid-addition salts are chosen from hydrochlorides, hydrobromides, sulphates, citrates, succinates, tartrates, lactates and acetates.

12. The composition according to claim 6, wherein said acid-addition salts are chosen from hydrochlorides, hydrobromides, sulphates, citrates, succinates, tartrates, lactates and acetates.

13. The composition according to claim 7, wherein said acid-addition salts are chosen from hydrochlorides, hydrobromides, sulphates, citrates, succinates, tartrates, lactates and acetates.

14. The composition according to claim 8, wherein said acid-addition salts are chosen from hydrochlorides, hydrobromides, sulphates, citrates, succinates, tartrates, lactates and acetates.

15. The composition according to claim 1, wherein said at least one first oxidation base represents from 0.0005 to 12% by weight relative to the total weight of the composition.

16. The composition according to claim 15, wherein said at least one first oxidation base represents from 0.005 to 6% by weight relative to the total weight of the composition.

17. The composition according to claim 1, wherein said at least one second oxidation base represents from 0.0005 to 12% by weight relative to the total weight of the composition.

18. The composition according to claim 17, wherein said at least one second oxidation base represents from 0.005 to 6% by weight relative to the total weight of the composition.

19. The composition according to claim 1, wherein said at least one coupler represents from 0.0001 to 10% by weight relative to the total weight of the composition.

20. The composition according to claim 19, wherein said at least one coupler represents from 0.005 to 5% by weight relative to the total weight of the composition.

21. The composition according to claim 1, further comprising water or a mixture of water and at least one organic solvent.

22. The composition according to claim 21, wherein said at least one organic solvent is chosen from C₁-C₄ alkanols, glycerol, glycols, glycol ethers, and aromatic alcohols.

23. The composition according to claim 22, wherein said at least one organic solvent is chosen from ethanol, isopropanol, 2-butoxyethanol, propylene glycol, propylene glycol monomethyl ether, diethylene glycol monoethyl ether, diethylene glycol monomethyl ether, benzyl alcohol, and phenoxyethanol.

24. The composition according to claim 21, wherein said at least one organic solvent represents from 1 to 40% by weight relative to the total weight of the composition.

25. The composition according to claim 24, wherein said at least one organic solvent represents from 5 to 30% by weight relative to the total weight of the composition.

26. The composition according to claim 1, further comprising at least one cosmetic adjuvant.

27. The composition according to claim 26, wherein said at least one cosmetic adjuvant is chosen from anionic, cationic, nonionic, amphoteric and zwitterionic surfactants; anionic, cationic, nonionic, amphoteric and zwitterionic polymers; inorganic and organic thickeners; antioxidants; penetration agents; sequestering agents; fragrances; buffers; dispersing agents; conditioners; film-forming agents; ceramides; sunscreens; preserving agents; and opacifiers.

28. A composition for oxidation dyeing of keratin fibers comprising: 1,8-bis(2,5-diaminophenoxy)-3,6-dioxaoctane tetrahydrochloride monohydrate, para-phenylenediamine, 5-N-(β-hydroxyethyl)amino-2-methylphenol, ethyl alcohol, sodium metabisulphite, pentasodium diethylenetriaminepentaacetic acid, aqueous ammonia, and water.

29. A composition for oxidation dyeing of keratin fibers comprising: 1,8-bis(2,5-diaminophenoxy)-3,6-dioxaoctane tetrahydrochloride monohydrate, para-phenylenediamine, 5-N-(β-hydroxyethyl)amino-2-methylphenol, ethanol, dipotassium hydrogenphosphate, potassium dihydrogenphosphate, sodium metabisulphite, pentasodium diethylenetriaminepentaacetic acid, and water.

30. A composition for oxidation dyeing of keratin fibers comprising
at least one oxidation base chosen from acid-addition salts of 1,8-bis(2,5-diaminophenoxy)-3,6-dioxaoctane, wherein said salts are chosen from hydrochlorides, hydrobromides, sulphates, citrates, succinates, tartrates, lactates and acetates;
at least one second oxidation base chosen from para-phenylenediamine, para-toluenediamine, N,N-bis-(β-hydroxyethyl)-para-phenylenediamine, 2-(β-hydroxyethyl)-para-phenylenediamine, 2,6-dimethyl-para-phenylenediamine, 2-isopropyl-para-phenylenediamine, 2-chloro-para-phenylenediamine, N-phenyl-para-phenylenediamine, 4,4′-diaminodiphenylamine, N-methoxyethyl-para-phenylenediamine, 2-n-propyl-para-phenylenediamine, 4-aminophenol, N-methyl-4-aminophenol, 2-hydroxymethyl-4-aminophenol, 3-methyl-4-aminophenol, 2-aminomethyl-4-aminophenol, 2-(p-hydroxyethylaminomethyl)-4-aminophenol, 2-methoxy-4-aminophenol, 2-methoxymethyl-4-aminophenol, tetraaminopyrimidine, 4-hydroxy-2,5,6-triaminopyrimidine, 4,5-diamino-1-ethyl-3-methylpyrazole, 4,5-diamino-N-methylpyrazole, 4,5-diamino-1-(4′-chlorobenzyl)pyrazole, N,N′-bis(β-hydroxyethyl)-N,N′-bis(4′-aminophenyl)-1,3-diaminopropanol, 3-amino-6-dimethylaminopyridine and pyrazolo[1,5-a]pyrimidines of formula (I): 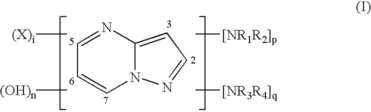
in which:
R₁, R₂, R₃ and R₄, which are identical or different, are chosen from a hydrogen atom, C₁-C₄ alkyl radicals, aryl radicals, C₁-C₄ hydroxyalkyl radicals, C₂-C₄ polyhydroxyalkyl radicals, (C₁-C₄)alkoxy(C₁-C₄)alkyl radicals, C₁-C₄ aminoalkyl radicals wherein said amino can be protected with a protective group chosen from acetyl, ureido and sulphonyl groups, (C₁-C₄)alkylamino(C₁-C₄)alkyl radicals, di((C₁-C₄)alkyl)amino(C₁-C₄)alkyl radicals, wherein said dialkyls can form a ring chosen from 5- and 6-membered aliphatic and heterocyclic rings, hydroxy(C₁-C₄)alkylamino(C₁-C₄)alkyl radicals, and di(hydroxy(C₁-C₄)alkyl)amino-(C₁-C₄)alkyl radicals;
radicals X are identical or different, and are chosen from a hydrogen atom, C₁-C₄ alkyl radicals, aryl radicals, C₁-C₄ hydroxyalkyl radicals, C₂-C₄ polyhydroxyalkyl radicals, C₁-C₄ aminoalkyl radicals, (C₁-C₄)alkylamino(C₁-C₄)alkyl radicals, di((C₁-C₄)alkyl)amino(C₁-C₄)alkyl radicals, wherein said dialkyls can form a ring chosen from 5- and 6-membered aliphatic and heterocyclic rings, hydroxy(C₁-C₄)alkylamino(C₁-C₄)alkyl radicals, di(hydroxy(C₁-C₄)alkyl)amino (C₁-C₄)alkyl radicals, amino radicals, (C₁-C₄)alkyl-amino radicals, di((C₁-C₄)alkyl)amino radicals, halogen atoms, carboxylic acid groups and sulphonic acid groups;
i is chosen from 0, 1, 2 and 3;
p is chosen from 0 and 1;
q is chosen from 0 and 1;
n is chosen from 0 and 1;
with the proviso that:
(i) the sum p+q is other than 0;
(ii) when p+q is equal to 2, then n is 0 and the groups NR₁R₂ and NR₃R₄ occupy positions (2,3); (5,6); (6,7); (3,5) and (3,7);
(iii) when p is equal to 1 and q is equal to 0, then n is 1 and the group NR₁R₂ and the OH group occupy positions (2,3); (5,6); (6,7); (3,5) and (3,7);
(iv) when p is equal to 0 and q is equal to 1, then n is 1 and the group NR₃R₄ and the OH group occupy positions (2,3); (5,6); (6,7); (3,5) and (3,7);
and acid-addition salts thereof;
and at least one coupler chosen from meta-phenylenediamines, meta-aminophenols, meta-diphenols, heterocyclic couplers, sesamol, a-naphthol, and acid-addition salts thereof.

31. The composition according to claim 30, wherein
said pyrazolo[1,5-a]pyrimidines of formula (I) are chosen from
pyrazolo[1,5-a]pyrimidine-3,7-diamine;
2-methylpyrazolo[1,5-a]pyrimidine-3,7-diamine;
2,5-dimethylpyrazolo[1,5-a]pyrimidine-3,7-diamine;
pyrazolo[1,5-a]pyrimidine-3,5-diamine;
2,7-dimethylpyrazolo[1,5-a]pyrimidine-3,5-diamine;
3-aminopyrazolo[1,5-a]pyrimidin-7-ol;
3-amino-5-methylpyrazolo[1,5-a]pyrimidin-7-ol;
3-aminopyrazolo[1,5-a]pyrimidin-5-ol;
2-(3-aminopyrazolo[1,5-a]pyrimidin-7-ylamino)ethanol;
3-amino-7-β-hydroxyethylamino-5-methylpyrazolo[1,5-a]pyrimidine;
2-(7-aminopyrazolo[1,5-a]pyrimidin-3-ylamino)ethanol;
2-((3-aminopyrazolo[1,5-a]pyrimidin-7-yl)-(2-hydroxyethyl)amino)ethanol;
2-((7-aminopyrazolo[1,5-a]pyrimidin-3-yl)-(2-hydroxyethyl)amino)ethanol;
5,6-dimethylpyrazolo[1,5-a]pyrimidine-3,7-diamine;
2,6-dimethylpyrazolo[1,5-a]pyrimidine-3,7-diamine;
2,5, N-7, N-7-tetramethylpyrazolo[1,5-a]pyrimidine-3,7-diamine;
and acid-addition salts thereof; and
said at least one coupler is chosen from
5-amino-2-methylphenol, 5-N-(β-hydroxyethyl)amino-2-methylphenol, 3-aminophenol, 1,3-dihydroxybenzene, 1,3-dihydroxy-2-methylbenzene, 4-chloro-1,3-dihydroxybenzene, 2,4-diamino-1-(β-hydroxyethyloxy)benzene, 2-amino-4-(β-hydroxyethylamino)-1-methoxybenzene, 1,3-diaminobenzene, 1,3-bis(2,4-diaminophenoxy)propane, 6-hydroxyindole, 4-hydroxyindole, 4-hydroxy-N-methylindole, 6-hydroxyindoline, 6-hydroxybenzomorpholine, 2,6-dihydroxy-4-methylpyridine, 1-H-3-methylpyrazol-5-one, 1-phenyl-3-methylpyrazol-5-one, and acid-addition salts thereof.

32. The composition according to claim 1, further comprising at least one direct dye.

33. A process for oxidation dyeing of keratin fibers, comprising:
applying to keratin fibers to be dyed a dyeing composition;
developing a desired color in said keratin fibers with the aid of at least one oxidizing agent;
wherein said dyeing composition comprises:
at least one first oxidation base chosen from 1,8-bis(2,5-diaminophenoxy)-3,6-dioxaoctane and acid-addition salts thereof,
at least one second oxidation base chosen from para-phenylenediamine, para-toluenediamine, N,N-bis-(β-hydroxyethyl)-para-phenylenediamine, 2-(β-hydroxyethyl)-para-phenylenediamine, 2,6-dimethyl-para-phenylenediamine, 2-isopropyl-para-phenylenediamine, 2-chloro-para-phenylenediamine, N-phenyl-para-phenylenediamine, 4,4′-diaminodiphenylamine, N-methoxyethyl-para-phenylenediamine, 2-n-propyl-para-phenylenediamine, 4-aminophenol, N-methyl-4-aminophenol, 2-hydroxymethyl-4-aminophenol, 3-methyl-4-aminophenol, 2-aminomethyl-4-aminophenol, 2-(β-hydroxyethylaminomethyl)-4-aminophenol, 2-methoxy-4-aminophenol, 2-methoxymethyl-4-aminophenol, tetraaminopyrimidine, 4-hydroxy-2,5,6-triaminopyrimidine, 4,5-diamino-1-ethyl-3-methylpyrazole, 4,5-diamino-N-methylpyrazole, 4,5-diamino-1-(4′-chlorobenzyl)pyrazole, N,N′-bis(β-hydroxyethyl)-N,N′-bis(4′-aminophenyl)-1,3-diaminopropanol, 3-amino-6-dimethylaminopyridine and pyrazolo[1,5-a]pyrimidines of formula (I): 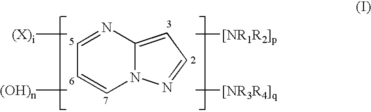
in which:
R₁, R₂, R₃ and R₄, which are identical or different, are chosen from a hydrogen atom, C₁-C₄ alkyl radicals, aryl radicals, C₁-C₄ hydroxyalkyl radicals, C₂-C₄ polyhydroxyalkyl radicals, (C₁-C₄)alkoxy(C₁-C₄)alkyl radicals, C₁-C₄ aminoalkyl radicals wherein said amino can be protected with a protective group chosen from acetyl, ureido and sulphonyl groups, (C₁-C₄)alkylamino(C₁-C₄)alkyl radicals, di((C₁-C₄)alkyl)amino(C₁-C₄)alkyl radicals, wherein said dialkyls can form a ring chosen from 5- and 6-membered aliphatic and heterocyclic rings, hydroxy(C₁-C₄)alkylamino(C₁-C₄)alkyl radicals, and di(hydroxy(C₁-C₄)alkyl)amino-(C₁-C₄)alkyl radicals;
radicals X are identical or different, and are chosen from a hydrogen atom, C₁-C₄ alkyl radicals, aryl radicals, C₁-C₄ hydroxyalkyl radicals, C₂-C₄ polyhydroxyalkyl radicals, C₁-C₄ aminoalkyl radicals, (C₁-C₄)alkylamino(C₁-C₄)alkyl radicals, di((C₁-C₄)alkyl)amino(C₁-C₄)alkyl radicals, wherein said dialkyls can form a ring chosen from 5- and 6-membered aliphatic and heterocyclic rings, hydroxy(C₁-C₄)alkylamino(C₁-C₄)alkyl radicals, di(hydroxy(C₁-C₄)alkyl)amino(C₁-C₄)alkyl radicals, amino radicals, (C₁-C₄)alkyl-amino radicals, di((C₁-C₄)alkyl)amino radicals, halogen atoms, carboxylic acid groups and sulphonic acid groups;
i is chosen from 0, 1, 2 and 3;
p is chosen from 0 and 1;
q is chosen from 0 and 1;
n is chosen from 0 and 1;
with the proviso that:
(i) the sum p+q is other than 0;
(ii) when p+q is equal to 2, then n is 0 and the groups NR₁R₂ and NR₃R₄ occupy positions (2,3); (5,6); (6,7); (3,5) and (3,7);
(iii) when p is equal to 1 and q is equal to 0, then n is 1 and the group NR₁R₂ and the OH group occupy positions (2,3); (5,6); (6,7); (3,5) and (3,7);
(iv) when p is equal to 0 and q is equal to 1, then n is 1 and the group NR₃R₄ and the OH group occupy positions (2,3); (5,6); (6,7); (3,5) and (3,7);
and acid-addition salts thereof;
and at least one coupler.

34. The process according to claim 33, wherein said dyeing composition further comprises at least one acidifying agent.

35. The process according to claim 34, wherein said at least one acidifying agent is chosen from inorganic acids, organic acids, carboxylic acids, and sulphonic acids.

36. The process according to claim 34, wherein said at least one acidifying agent is chosen from hydrochloric acid, orthophosphoric acid, sulphuric acid, acetic acid, tartaric acid, citric acid and lactic acid.

37. The process according to claim 33, wherein said dyeing composition further comprises at least one basifying agent.

38. The process according to claim 37, wherein said at least one basifying agent is chosen from aqueous ammonia, alkaline carbonates, alkanolamines, sodium hydroxide, potassium hydroxide, and compounds of formula (II): 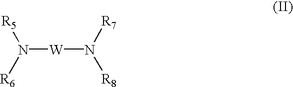
in which
W is a propylene residue optionally having a substituent chosen from a hydroxyl group and C₁-C₆ alkyl radicals; R₅, R₆, R₇ and R₈, which may be identical or different, are chosen from a hydrogen atom, C₁-C₆ alkyl and C₁-C₆ hydroxyalkyl radicals.

39. The process according to claim 33, wherein said developing occurs at an acidic pH.

40. The process according to claim 39, wherein said acidic pH ranges from 3 to 5.7.

41. The process according to claim 33, wherein said developing occurs at a neutral pH.

42. The process according to claim 33, wherein said developing occurs at an alkaline pH.

43. The process according to claim 42, wherein said alkaline pH is 10.

44. The process according to claim 33, wherein said at least one oxidizing agent is added to said dyeing composition just prior to said applying of said dyeing composition.

45. The process according to claim 33, wherein said at least one oxidizing agent is present in an oxidizing composition.

46. The process according to claim 45, wherein said oxidizing composition is applied to said keratin fibers simultaneously with said applying of said dyeing composition.

47. The process according to claim 45, wherein said oxidizing composition is applied to said keratin fibers after said applying of said dyeing composition.

48. The process according to claim 45, wherein said oxidizing composition has a pH ranging from 3 to 12.

49. The process according to claim 48, wherein said oxidizing composition has a pH ranging from 5 to 11.

50. The process according to claim 45, wherein said oxidizing composition further comprises at least one cosmetic adjuvant.

51. The process according to claim 50, wherein said at least one cosmetic adjuvant is chosen from anionic, cationic, nonionic, amphoteric and zwitterionic surfactants; anionic, cationic, nonionic, amphoteric and zwitterionic polymers; inorganic and organic thickeners; antioxidants; penetration agents; sequestering agents; fragrances; buffers; dispersing agents; conditioners; film-forming agents; ceramides; sunscreens; preserving agents; and opacifiers.

52. The process according to claim 33, wherein said at least one oxidizing agent is chosen from hydrogen peroxide, urea peroxide, alkali metal bromates, persalts and enzymes.

53. The process according to claim 52, wherein said at least one oxidizing agent is chosen from perborates, persulphates, peroxidases, 2-electron oxidoreductases, and 4-electron oxygenases.

54. The process according to claim 52, wherein said at least one oxidizing agent is chosen from uricases and lactases.

55. A multi-compartment dyeing device, comprising:
a first compartment,
a second compartment;
wherein said first compartment contains a dyeing composition comprising:
at least one first oxidation base chosen from 1,8-bis(2,5-diaminophenoxy)-3,6-dioxaoctane and acid-addition salts thereof;
at least one second oxidation base chosen from para-phenylenediamine, para-toluenediamine, N,N-bis-(β-hydroxyethyl)-para-phenylenediamine, 2-(β-hydroxyethyl)-para-phenylenediamine, 2,6-dimethyl-para-phenylenediamine, 2-isopropyl-para-phenylenediamine, 2-chloro-para-phenylenediamine, N-phenyl-para-phenylenediamine, 4,4′-diaminodiphenylamine, N-methoxyethyl-para-phenylenediamine, 2-n-propyl-para-phenylenediamine, 4-aminophenol, N-methyl-4-aminophenol, 2-hydroxymethyl-4-aminophenol, 3-methyl-4-aminophenol, 2-aminomethyl-4-aminophenol, 2-(β-hydroxyethylaminomethyl)-4-aminophenol, 2-methoxy-4-aminophenol, 2-methoxymethyl-4-aminophenol, tetraaminopyrimidine, 4-hydroxy-2,5,6-triaminopyrimidine, 4,5-diamino-1-ethyl-3-methylpyrazole 4,5-diamino-N-methylpyrazole, 4,5-diamino-1-(4′-chlorobenzyl)pyrazole, N,N′-bis(β-hydroxyethyl)-N,N′-bis(4′-aminophenyl)-1,3-diaminopropanol, 3-amino-6-dimethylaminopyridine and pyrazolo[1,5-a]pyrimidines of formula (I): 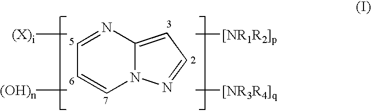
in which:
R₁, R₂, R₃ and R₄, which are identical or different, are chosen from a hydrogen atom, C₁-C₄ alkyl radicals, aryl radicals, C₁-C₄ hydroxyalkyl radicals, C₂-C₄ polyhydroxyalkyl radicals, (C₁-C₄)alkoxy(C₁-C₄)alkyl radicals, C₁-C₄ aminoalkyl radicals wherein said amino can be protected with a protective group chosen from acetyl, ureido and sulphonyl groups, (C₁-C₄)alkylamino(C₁-C₄)alkyl radicals, di((C₁-C₄)alkyl)amino(C₁-C₄)alkyl radicals, wherein said dialkyls can form a ring chosen from 5- and 6-membered aliphatic and heterocyclic rings, hydroxy(C₁ -C₄)alkylamino(C₁-C₄)alkyl radicals, and di(hydroxy(C₁-C₄)alkyl)amino-(C₁-C₄)alkyl radicals;
radicals X are identical or different, and are chosen from a hydrogen atom, C₁-C₄ alkyl radicals, aryl radicals, C₁-C₄ hydroxyalkyl radicals, C₂-C₄ polyhydroxyalkyl radicals, C₁-C₄ aminoalkyl radicals, (C₁-C₄)alkylamino(C₁-C₄)alkyl radicals, di((C₁-C₄)alkyl)amino(C₁-C₄)alkyl radicals, wherein said dialkyls can form a ring chosen from 5- and 6-membered aliphatic and heterocyclic rings, hydroxy(C₁-C₄)alkylamino(C₁-C₄)alkyl radicals, di(hydroxy(C₁-C₄)alkyl)amino(C₁-C₄)alkyl radicals, amino radicals, (C₁-C₄)alkyl-amino radicals, di((C₁-C₄)alkyl)amino radicals, halogen atoms, carboxylic acid groups and sulphonic acid groups;
i is chosen from 0, 1, 2 and 3;
p is chosen from 0 and 1;
q is chosen from 0 and 1;
n is chosen from 0 and 1;
with the proviso that:
(i) the sum p+q is other than 0;
(ii) when p+q is equal to 2, then n is 0 and the groups NR₁R₂ and NR₃R₄ occupy positions (2,3); (5,6); (6,7); (3,5) and (3,7);
(iii) when p is equal to 1 and q is equal to 0, then n is I and the group NR₁R₂ and the OH group occupy positions (2,3); (5,6); (6,7); (3,5) and (3,7);
(iv) when p is equal to 0 and q is equal to 1, then n is 1 and the group NR₃R₄ and the OH group occupy positions (2,3); (5,6); (6,7); (3,5) and (3,7);
and acid-addition salts thereof;
and at least one coupler;
wherein said second compartment contains an oxidizing composition comprising:
at least one oxidizing agent.

56. A dyeing kit comprising:
a first container,
a second container;
wherein said first container contains a dyeing composition comprising:
at least one first oxidation base chosen from 1,8-bis(2,5-diaminophenoxy)-3,6-dioxaoctane and acid-addition salts thereof;
at least one second oxidation base chosen from para-phenylenediamine, para-toluenediamine, N,N-bis-(β-hydroxyethyl)-para-phenylenediamine, 2-(β-hydroxyethyl)-para-phenylenediamine, 2,6-dimethyl-para-phenylenediamine, 2-isopropyl-para-phenylenediamine, 2-chloro-para-phenylenediamine, N-phenyl-para-phenylenediamine, 4,4′-diaminodiphenylamine, N-methoxyethyl-para-phenylenediamine, 2-n-propyl-para-phenylenediamine, 4-aminophenol, N-methyl-4-aminophenol, 2-hydroxymethyl-4-aminophenol, 3-methyl-4-aminophenol, 2-aminomethyl-4-aminophenol, 2-(β-hydroxyethylaminomethyl)-4-aminophenol, 2-methoxy-4-aminophenol, 2-methoxymethyl-4-aminophenol, tetraaminopyrimidine, 4-hydroxy-2,5,6-triaminopyrimidine, 4,5-diamino-1-ethyl-3-methylpyrazole, 4,5-diamino-N-methylpyrazole, 4,5-diamino-1-(4′-chlorobenzyl)pyrazole, N,N′-bis(β-hydroxyethyl)-N,N′-bis(4′-aminophenyl)-1,3-diaminopropanol, 3-amino-6-dimethylaminopyridine and pyrazolo[1,5-a]pyrimidines of formula (I): 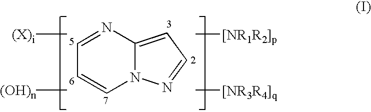
in which:
R₁, R₂, R₃ and R₄, which are identical or different, are chosen from a hydrogen atom, C₁-C₄ alkyl radicals, aryl radicals, C₁-C₄ hydroxyalkyl radicals, C₂-C₄ polyhydroxyalkyl radicals, (C₁-C₄)alkoxy(C₁-C₄)alkyl radicals, C₁-C₄ aminoalkyl radicals wherein said amino can be protected with a protective group chosen from acetyl, ureido and sulphonyl groups, (C₁-C₄)alkylamino(C₁-C₄)alkyl radicals, di((C₁-C₄)alkyl)amino(C₁-C₄)alkyl radicals, wherein said dialkyls can form a ring chosen from 5- and 6-membered aliphatic and heterocyclic rings, hydroxy( C₁-C₄)alkylamino(C₁-C₄)alkyl radicals, and di(hydroxy(C₁-C₄)alkyl)amino-(C₁-C₄)alkyl radicals;
radicals X are identical or different, and are chosen from a hydrogen atom, C₁-C₄ alkyl radicals, aryl radicals, C₁-C₄ hydroxyalkyl radicals, C₂-C₄ polyhydroxyalkyl radicals, C₁-C₄ aminoalkyl radicals, (C₁-C₄)alkylamino(C₁-C₄)alkyl radicals, di((C₁-C₄)alkyl)amino(C₁-C₄)alkyl radicals, wherein said dialkyls can form a ring chosen from 5- and 6-membered aliphatic and heterocyclic rings, hydroxy(C₁-C₄)alkylamino(C₁-C₄)alkyl radicals, di(hydroxy(C₁ -C₄)alkyl)amino(C₁-C₄)alkyl radicals, amino radicals, (C₁-C₄)alkyl-amino radicals, di((C₁-C₄)alkyl)amino radicals, halogen atoms, carboxylic acid groups and sulphonic acid groups;
i is chosen from 0, 1, 2 and 3;
p is chosen from and 1;
q is chosen from 0 and 1;
n is chosen from 0 and 1;
with the proviso that:
(i) the sum p+q is other than 0;
(ii) when p+q is equal to 2, then n is 0 and the groups NR₁,R₂ and NR₃R₄ occupy positions (2,3); (5,6); (6,7); (3,5) and (3,7);
(iii) when p is equal to 1 and q is equal to 0, then n is 1 and the group NR₁R₂ and the OH group occupy positions (2,3); (5,6); (6,7); (3,5) and (3,7);
(iv) when p is equal to 0 and q is equal to 1, then n is I and the group NR₃R₄ and the OH group occupy positions (2,3); (5,6); (6,7); (3,5) and (3,7);
and acid-addition salts thereof;
and at least one coupler;
wherein said second container contains an oxidizing composition comprising:
at least one oxidizing agent.
